(19) United States
(12) Patent Application Publication
(10) Pub. No.: US 2005/0211922 A1
(43) Pub. Date: Sep. 29, 2005

(54) Minute three dimensional structure producing apparatus and method

(76) Inventors: Masanao Munekane, Chiba-shi (JP); Kouji Iwasaki, Chiba-shi (JP)

Correspondence Address: BRUCE L. ADAMS, ESQ.;31ST FLOOR, 50 BROADWAY, NEW YORK, NY, 10004, US

(21) Appl. No.: 11/086,492

(22) Filed: Mar. 22, 2005

(30) Foreign Application Priority Data
Mar. 23, 2004 (JP) 2004-084773
Mar. 15, 2005 (JP) 2005-073315

(51) Int. Cl. A61N 5/00; G21G 5/00

(52) U.S. Cl. 250/492.3; 250/492.21

(57) ABSTRACT

A structure having arbitrary rotational symmetry is produced by attaching a sample stage (turntable) to a precision rotational shaft that is continuously rotated as high precision, performing FIB deposition inside an FIB chamber while causing continuous rotation of the sample stage, or performing cut-way processing from a side surface or upper surface, like a general purpose lathe, using FIB etching.

### BACKGROUND OF THE INVENTION

The present invention relates to a focused ion beam (FIB) device for making a minute three-dimensional structure of a micro-level, or a minute three-dimensional structure manufacturing device using a scanning electron microscope (SEM).

Turning lathes are mainly used for circular processing or axial processing, with a method that has been used for a long time.

Generally, it is possible to form an arbitrary shape that is rotationally symmetrical by pressing a hard blade down and cutting. In actual fact, with respect to technology for forming a micro level or nanoscale structure with a lathe, this is not achieved, and therefore it has not been considered. Patent publication 1 discloses recommendations for a precise turntable and accurate measurement, but there are a lot of proposals besides this, and a mechanism for suppressing axial play below a micro-level and correction of this are discussed.

Conventionally, in a processing method using an FIB, a method accompanied by rotation of the sample was not selected, and in the case of making a circular object, scanning of the FIB beam is carried out in a circular scan pattern, and scanning is carried out to reflect a circular image with a raster scan. For this reason, in the case of circular processing, smooth processing is difficult.

Patent publication 2 shows a mechanism for rotating an arbitrary sample having a rotational axis in a vertical direction with respect to an energy beam axis, and correcting positional displacement of a center of rotation by measuring a deflection reference axis. However, conditions where precision of axial play is improved are not considered, and processing with highly accurate or high speed rotation is not mentioned. When performing processing, in order to carry out correction and processing after rotating to an arbitrary angle, smooth circular processing is not possible, and when performing processing in a smooth shape, there is a problem that a lot of processes are required.

[Patent Publication 1] Japanese Patent Laid-open No. Hei. 11-311511, specification “Ultra Precise Revolver” (Page 8, FIG. 1).

[Patent Publication 2] Japanese Patent Laid-open No Hei. 3-280342, specification “Three Dimensional processing device for minute part”, (page 5, FIG. 3).

The object of the present invention is to solve the above described problems, and to provide a minute three dimensional structure producing apparatus and method, capable of manufacturing a minute three dimensional structure that has rotational symmetry and a smooth surface, easily, with high throughput and with high precision, using deposition or etching utilizing a charged particle beam.

### SUMMARY OF THE INVENTION

In order to achieve the above described object, the minute three dimensional structure producing device of this application comprises a vacuum chamber, a turntable provided in the vacuum chamber, charged particle beam irradiating means for irradiating charged particle beam on the turntable, a gas gun for supplying a gas for deposition at the charged particle beam irradiated position on the turntable, rotating means for rotating the turntable continuously, so that a minute three dimensional structure having rotational symmetry is produced by depositing a film on the turntable while rotating the turntable continuously during supplying the gas for deposition.

A focused ion beam or a electron beam is used for the charged particle beam.

Further, the minute three-dimensional structure producing device according to the present invention comprises a vacuum chamber, a turntable provided in the vacuum chamber, charged particle beam irradiating means for irradiating charged particle beam on a sample disposed on the turntable, rotating means for rotating the turntable continuously, so that a minute three dimensional structure having rotational symmetry is produced by etching the sample while rotating the turntable continuously during irradiating the charged particle beam on the sample.

The etching is a gas assisted etching or sputter etching.

Further, the minute three-dimensional structure producing device according to the present invention comprises a vacuum chamber, a turntable provided in the chamber for rotating a sample on it, a microprobe for processing the sample by pressing the rotating sample, and a fine movement element for moving the microprobe three-dimensionally.

A minute three dimensional structure producing method according to the present invention forms a rotationally symmetrical three dimensionally shaped structure by depositing a film on a sample stage using deposition using a charged particle beam while causing continuous rotation of the sample stage.

In the minute three dimensional structure producing method, the three dimensionally shaped structure is formed at a rotationally central place on the sample stage, and the three dimensionally shaped structure is used as a processing tool.

In the minute three dimensional structure producing method according to the present invention, the processing tool has a drill bit shape, and is used as a hole forming tool by being rotated.

In the minute three dimensional structure producing method, an object in which a hole is to be formed is placed on the hole forming tool using a manipulator that is moved with precision by a piezo driver, and hole formation processing is carried out by rotating the sample stage.

In the minute three dimensional structure producing method, in the case of irradiation where the position of an FIB spot beam is fixed away from a rotational axis center and irradiated while continuously rotating a turntable, a circular locus is traced accompanying rotation of the turntable, and positioning of the rotational center of the turntable is carried out from the circular locus.

In the minute three dimensional structure producing method, rotational axis shift is estimated by comparing the diameter of the charged particle beam and a processing width of a circular processing locus drawn when irradiating the charged particle beam with a position of the charged particle beam fixed to a place on a turntable away from the rotational center, and processing is carried out by selecting a beam diameter matching a desired rotation precision.

With the minute three dimensional structure producing machine of the present invention, by providing a rotation mechanism for continuously rotating a sample stage (turntable 1), and using a device that has excellent rotational precision of a rotating shaft of the rotation mechanism, it is possible to easily produce a minute three dimensional structure that has rotational symmetry with good precision by deposition or etching with a charged particle beam. It is also possible to repeat creation/correction of rotationally symmetrical structures using a combination of deposition of structural shapes using a charged particle beam and lathe processing by etching, and it is possible to perform accurate processing compared to processing using only deposition. It is also possible to carry out smooth processing in a circular shape that does not require correction every time a conventional rotational shaft is turned, by combining etching and rotation.

Also, a processed sample is rotated with high precision, which means that it is possible to make use of shape and utilize as a rotational device mechanism that is capable of having a hole forming function such as a drill.

Also, as shown in FIG. 3, it becomes easy to make a reverse tapered structure 11 that widens out in a reverse direction, which was impossible with conventional deposition, and if a rotational shaft of a turntable 1 is made orthogonal with respect to an axis of a focused ion beam, isolation of a lower part of a sample is possible by etching directly from the side, it is possible to isolate a sample with a combination of manipulators and handling is possible.

### BRIEF DESCRIPTION OF THE DRAWINGS

FIG. 1 is a schematic diagram of a turntable.

FIG. 2 is a drawing showing arrangement of the turntable inside an FIB chamber.

FIG. 3 is an explanatory drawing for a structure having a reverse taper angle

FIG. 4 is a perspective view showing mother body formation using deposition.

FIG. 5 is a perspective view showing circular cutting processing using processing from the side.

FIG. 6 is a perspective view showing a cylindrical mother body formed by cutting while rotating.

FIG. 7 is a perspective view showing a wineglass formed using the process of FIG. 5.

FIG. 8 is a perspective view showing a nanodrill formation process using deposition.

FIG. 9 is a perspective view showing an example of a formed nanodrill.

FIG. 10 is a perspective view showing before hole formation.

FIG. 11 is a perspective view showing after hole formation.

### DETAILED DESCRIPTION OF THE INVENTION

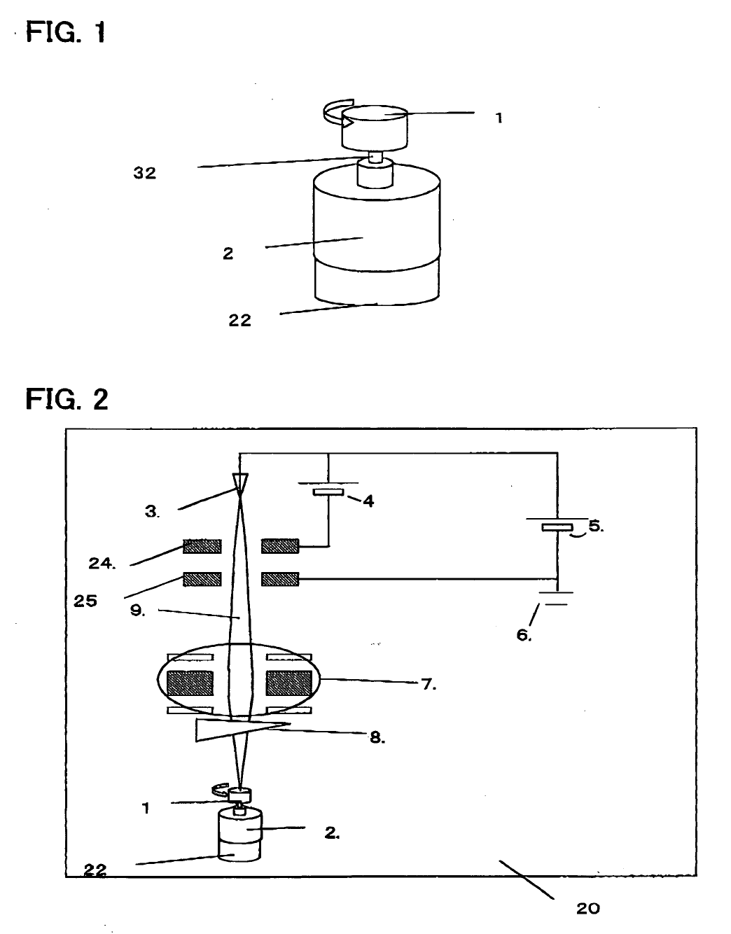

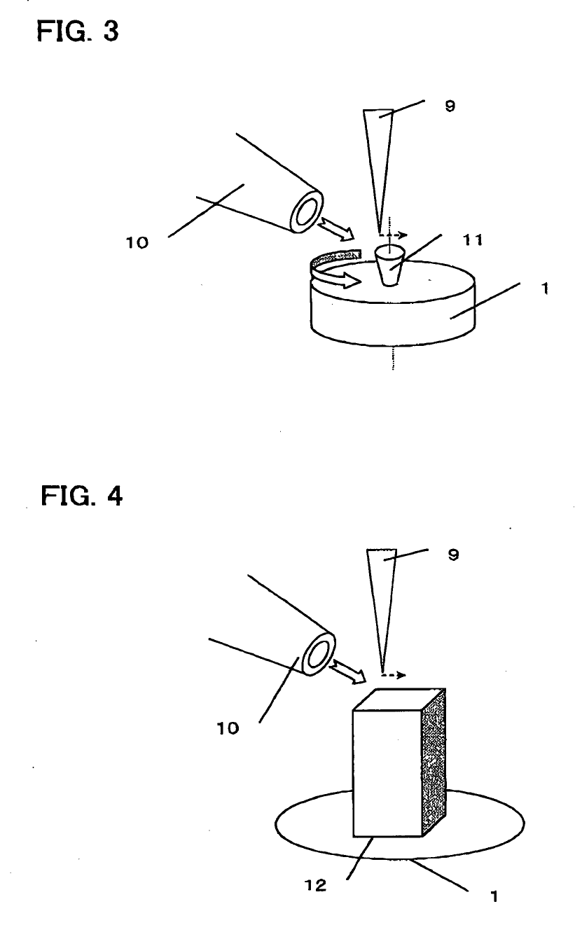

The minute three dimensional structure producing device of this application is provided with rotation shaft 32 having a high precision axle bearing, connected to a sample stage (turntable) inside a vacuum chamber of a FIB device or SEM device and rotating means for causing continuous rotation of the precision rotation shaft. FIG. 1 shows a schematic view of the turntable used in the present minute three dimensional structure producing device. Specifically, as shown in FIG. 1, it is possible to rotate a precision rotation shaft 32 having a low play precision axle bearing 2 inside a vacuum chamber 20 using a rotational drive mechanism like motor 22. FIG. 2 shows a Focused ion beam apparatus which is the minute three dimensional structure producing device using the turntable. Ion beam extracted from ion source 3 by extraction electrode 24 to which extraction voltage 4 is applied is accelerated by acceleration electrode 25 to which acceleration electrode 5 is applied. The accelerated ion beam is condensed by lens 7. The condensed ion beam is scanned by deflector 8 and irradiated at a arbitrary position of the sample on turntable 1. Number 6 denotes ground. As shown in FIG. 2, turntable 1 fixed to this precision rotational shaft is used as a processing tool caused to rotate inside an FIB chamber. For example, by carrying out deposition or etching in a platform that has a shaft play precision of less than 0.1 μm and continuously rotates at a high speed of 1 revolution per second or higher, it is possible to perform processing for an arbitrary three dimensional structure, such as a wheel, being a circular structure at a microlevel. Dimensional values shown here are one indication, it is preferable to be able to manufacture a three dimensional structure at a microlevel, and there is no particular limited meaning to these values.

By using the minute three dimensional structure producing device of the present invention, it is possible to manufacture a shape such as a wheel in the case of deposition while performing rotational processing causing continuous rotation of a sample stage (turntable) fixed to an precision rotational shaft, and in the case of etching, it is possible to manufacture a structure that is symmetrical by performing cutting processing such as a lathe from a side surface or upper surface while rotating. The sample stage itself preferably enables a user to monitor or process at an arbitrary angle, on a 5-axis stage (X, Y, Z, R: rotation, T: tilt).

With processing according to the present invention which is conducted rotating a rotational shaft, first of all the following method is used to obtain a rotational center of a turntable fixed to a precision rotational shaft with shaft play of less than 0.1 μm. In the case of irradiation where an FIB spot beam is positionally fixed and irradiated on a turntable while continuously rotating, with a rotational axis center slipped by R μm, since a circular locus is traced accompanying rotation of radius R, the rotational center is close to the center of the locus. This operation is repeated, if necessary, and the rotational center is narrowed down to within a range of 1 μm. Next, formation of a basic processing shape is started at the rotational center using FIB deposition processing, which is done by irradiation of a focused ion beam onto a sample while blowing source material gas there using a gas gun 10, and for that basic shape cutting processing is carried out from a side surface using FIB etching. In this case, cutting processing is carried out while causing the turntable to continuously rotate.

Also, with processing according to the present invention which is conducted rotating a rotational shaft, it is preferable to select a processing current value for the FIB in order to perform processing matching size of the processing object and shaft play. For example, with a beam diameter of 0.1 μm, an FIB spot beam is positionally fixed and irradiated onto a turntable in advance while causing continuous rotation. When line width of the processed circle locus which is formed by irradiating a beam to a place which is R μm away from the rotational shaft center is 0.6 μm, since there is rotational shaft play of 0.5 μm (±0.25 μm) at the time of rotation, beam current is selected to give a beam diameter for obtaining a desired line width taking into consideration an error of 0.5 μm. For example, if it is desired to carry out processing at a line width of 1 μm, since it is preferable to make beam diameter 0.5 μm, beam current corresponding to this beam diameter is selected. If the FIB processing current value is large, processing rate and beam diameter have a relationship becoming large, which means that appropriate selection of processing size, axial precision and beam current is effective.

Also, in the case where beam scan rate is extremely fast scanning of a few μ seconds per single processing region for a rotational speed of, for example, 1 rps when designating a processing region, processing is distributed over the entire processing frame, while on the other hand in the case of slow scanning for a rotational speed of 1 rps it is also possible to carry out shape processing corresponding to scan rate for helical structures, etc.

In this way, scanning for FIB etching and FIB deposition etc. is not limited to rectangular raster scanning, and it is also possible to use a system for processing an arbitrary shape by using a spot beam for irradiating a vector scan or beam to a fixed position or a bitmap, to thereby designate a beam scanning range for a raster scan and hold a shape.

Also, by changing a rotational axis of the turntable 1 to a horizontal or vertical direction with respect to the FIB beam axis, it is also possible to have the scan direction from an upper surface of a sample or from a side surface of a sample, and selection is made corresponding to a shape formation method that is desired for processing. Using processing from the side surface, a structure having a reverse taper angle, which could not be made with deposition processing from a direction above the sample in the related art can also be made using processing that performs etching of a mother body from a side surface.

Dimensional values shown here are one indication, it is preferable to be able to manufacture a three dimensional structure at a microlevel, and there is no particular limited meaning to these values.

Next, with rotational processing of the present invention, a structure that is an object of processing is mounted on the rotational center using a manipulator or the like, and similarly, it is possible to designate a region it is desired to subject to etching processing while causing rotation, and to carry out processing. The manipulator is a microprobe moving three XYZ axes with a piezo driver and moving minutely, a microprobe tip formed to a point is operated and moved, and has a function capable of fixing a sample by deposition and conveying it.

In the next step, a structure formed at the rotational center is rotated like a drill, and another sample is brought in by the manipulator, and by pressing against a blade edge, it is possible to form a hole without damage of irradiated ions on the sample which was a problem when forming holes in another sample through FIB etching.

In the case of circular processing, a stage is rotated in a stepwise manner, as in patent publication 2, and by carrying out processing using a plurality of steps while monitoring an SIM image or an SEM image, it is possible to form a sample body into a smooth shaped object corresponding to rotational precision better than with processing by rotating continuously on a precision axis. Also, by inclining the rotational axis of the turntable 1 (sample stage ) close to 90°, and carrying out etching processing from a sideways method, it is possible to cut away a lower part of a formed structure, and it is also possible to handle a three dimensional structure using a manipulator or the like.

The most significant characteristic of the above described invention is that it is possible to form a three dimensional structure having rotational symmetry using an FIB.

It is possible to monitor a structure and manufacturing process using an FIB with a scanning electron microscope (SEM). It is also possible to carry out continuous deposition operations with charged particle beam deposition instead of FIB.

### First Embodiment

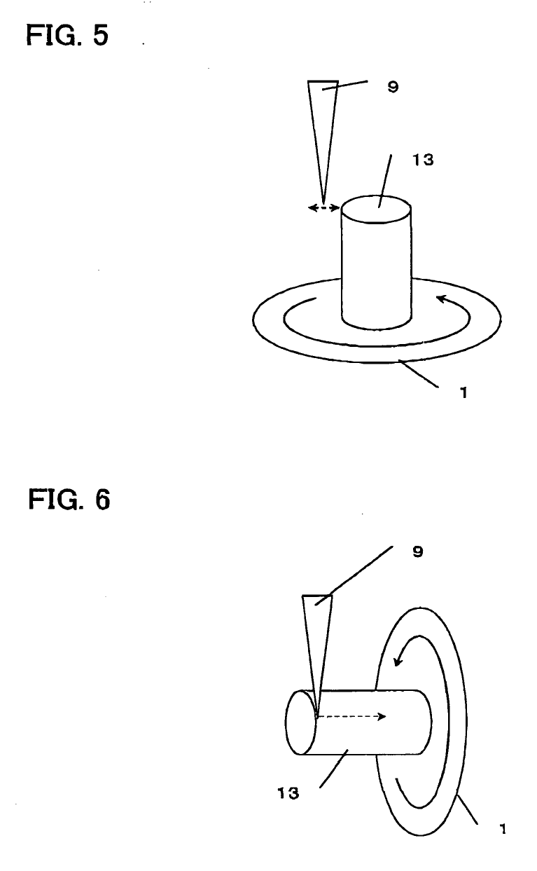

FIG. 4 is a perspective view showing formation of a base body using deposition. As shown in FIG. 4, the FIB beam 9 is irradiated from above, a shape matching a formed shape and a position close to rotational center on the turntable 1 are designated, assist gas is supplied using a gas gun 10, FIB deposition is carried out, and by depositing a deposition layer an arbitrary shape of base body 12 is formed. FIG. 5 is a perspective view showing cutting process of base body 12 into cylindrical shape. As shown in FIG. 5, while causing continuous rotation of the turntable 1, and while the focused ion beam 9 is scanned in the radial direction of the turntable 1, etching is carried out in a rotational axis direction and a cylindrical member 13 having rotational symmetry is formed.

### Second Embodiment

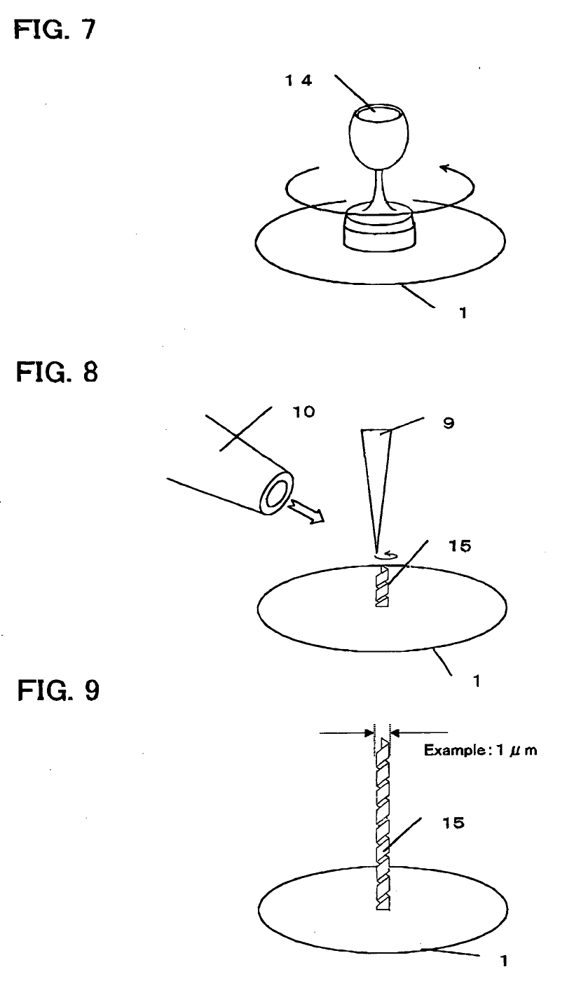

FIG. 6 is perspective view of cylindrical body 13 formed by cutting a base body during irradiating ion beam 9 from the side of the base body. FIG. 7 is a perspective view of wine glass 14 formed by using the process of FIG. 5 or FIG. 6. As shown in FIG. 6 and FIG. 7, it is possible to form an arbitrary shape having rotational symmetry such as a wineglass 14. Also, by performing etching while causing rotation of a lower part of a formed structure, isolation is possible, and it is also possible to handle a formed structure using a manipulator.

### Third Embodiment

While causing continuous rotation of an arbitrary sample that is brought in at a rotational center by a manipulator or the base body formed in embodiment 1, on the turntable 1, an arbitrary rotationally symmetrical shape is formed by removing a side surface or upper surface with a mechanism such as a lathe by scratching with a probe needle. Also, by scratching the root of a formed structure in the same way, isolation is possible, and the formed structure can be also be manipulated.

### Fourth Embodiment

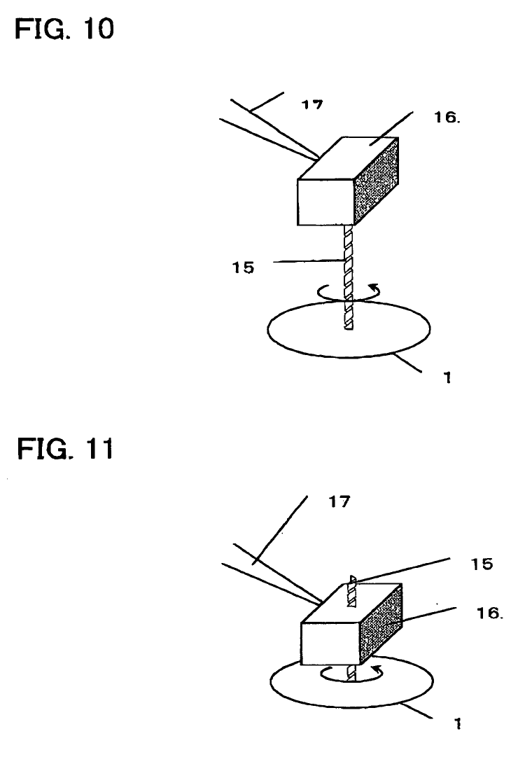

FIG. 8 is a perspective view showing a process of forming a nano-size-drill by deposition. FIG. 9 is a perspective view showing a nano-sized-drill formed by this process. As shown in FIG. 8 and FIG. 9, a drill bit 15 or pick shape having a diameter of 1 μm is formed by deposition or etching processing at a rotational center similarly to embodiment 1, and by causing an arbitrary sample as shown in FIG. 10 or FIG. 11 to approach on a drill bit using a precision handling mechanism such as a manipulator 17, a hole is formed while continuously rotating the turntable 1.

### Fifth Embodiment

A drill bit or pick shaped structure, similar to embodiment 4, is held by a micro handling tool such as a manipulator and fixed by a chucking mechanism or deposition, an arbitrary sample in which it is desired to form a hole is again made to approach by using the manipulator again, and a hole is formed while causing rotation. It is also possible for an initial member for making a drill bit to be held at a rotational center, and to process a cut-away blade shape by etching processing or the like.

As is clear from the above description, according to the present invention, application in the field of minute processing, such as future formation of minute devices, formation of arrays, and formation of optical devices, nanoimprint elements etc. has been conceived. If it is possible to produce structures having symmetry and being rounder, it is possible to use as a shape formation tool having a function called a lathe function in the field of mechanical engineering for structure formation. Also, if it is possible to form a minute drill on a rotational center and form holes by rotation/cutting away, it is possible to carry out hole formation processing with conventional semiconductor processing or an FIB, and there is no danger of contamination due to an irradiated ions, such as an FIB. In actual fact, application of defect formation of photonic crystal has been considered.

1. A minute three dimensional structure producing device comprises a vacuum chambers, a turntable provided in the vacuum chamber, charged particle beam irradiating means for irradiating charged particle beam on the turntable, a gas gun for supplying a gas for deposition at the charged particle beam irradiated position on the turntable, rotating means for rotating the turntable continuously, so that a minute three dimensional structure having rotational symmetry is produced by depositing a film on the turntable while rotating the turntable continuously during supplying the gas for deposition.

2. A minute three-dimensional structure producing device of claim 1, wherein the charged particle beam is a focused ion beam (FIB) or an electron beam.

3. A minute three-dimensional structure producing device comprises a vacuum chamber, a turntable provided in the vacuum chamber, charged particle beam irradiating means for irradiating charged particle beam on a sample disposed on the turntable, rotating means for rotating the, turntable continuously, so that a minute three dimensional structure having rotational symmetry is produced by etching the sample while rotating the turntable continuously during irradiating the charged particle beam on the sample.

4. A minute three-dimensional structure producing device of claim 3, wherein the charged particle beam is an FIB or an electron beam.

5. A minute three-dimensional structure producing device of claim 3, wherein the etching is gas assist etching.

6. A minute three-dimensional structure producing device comprises a vacuum chamber, a turntable provided in the chamber for rotating a sample on it, a microprobe for processing the sample by pressing the rotating sample, and a fine movement element for moving the microprobe three-dimensionally.

7. A minute three dimensional structure producing method which forms a rotationally symmetrical three dimensionally shaped structure by depositing a film on a sample stage using deposition using a charged particle beam while causing continuous rotation of the sample stage.

8. A minute three dimensional structure producing method of claim 7, wherein the three dimensionally shaped structure is formed at a rotationally central place on the sample stage, and the three dimensionally shaped structure is used as a processing tool.

9. A minute three dimensional structure producing method of claim 8, wherein the processing tool has a drill bit shape, and is used as a hole forming tool by being rotated.

10. A minute three dimensional structure producing method of claim 9, wherein an object in which a hole is to be formed is placed on the hole forming tool using a manipulator that is moved with precision by a piezo driver, and hole formation processing is carried out by rotating the sample stage.

11. A minute three dimensional structure producing method according to claim 7, wherein, in the case of irradiation where an FIB spot beam is positionally fixed away from a rotational axis center and irradiated while continuously rotating a turntable, a circular locus is traced accompanying rotation of the turntable, and positioning of the rotational center of the turntable is carried out from the circular locus.

12. A minute three dimensional structure producing method according to claim 7, wherein rotational axis shift is estimated by comparing the diameter of the charged particle beam and a processing width of a circular processing locus drawn when irradiating the charged particle beam with a position of the charged particle beam fixed to a place on a turntable away from the rotational center, and processing is carried out by selecting a beam diameter matching a desired rotation precision.
